(19) United States
(12) Patent Application Publication
(10) Pub. No.: US 2023/0304104 A1
(43) Pub. Date: Sep. 28, 2023

(54) CANCER DRUG SENSITIVITY DETERMINING MARKERS

(71) Applicant: Lantern Pharma Inc., Dallas, TX (US)

(72) Inventors: Kishor Bhatia, Ellicott City, MD (US); Aditya Kulkarni, Dallas, TX (US)

(21) Appl. No.: 18/319,919

(22) Filed: May 18, 2023

Related U.S. Application Data
(63) Continuation of application No. PCT/US2021/072496, filed on Nov. 18, 2021.
(60) Provisional application No. 63/115,580, filed on Nov. 18, 2020.
(60) Provisional application No. 63/127,102, filed on Dec. 17, 2020.
(60) Provisional application No. 63/223,540, filed on Jul. 19, 2021.

(51) Int. Cl. C12Q 1/6886

(52) U.S. Cl. CPC C12Q 1/6886; C12Q 2600/106; C12Q 2600/158

(57) ABSTRACT

This application relates to markers for use in the determination of the sensitivity of a cancer patient to an anti-cancer agent (e.g., illudins/illudin analogs) to be administered thereto, which markers can determine whether or not the cancer of the patient has a therapeutic response to the anti-cancer agent, and to application of the markers.

### CROSS-REFERENCE

This application claims the benefit of U.S. Provisional Application No. 63/115,580, filed Nov. 18, 2020, U.S. Provisional Application No. 63/127,102, filed Dec. 17, 2020, and U.S. Provisional Application No. 63/223,540, filed Jul. 19, 2021, all of which applications are incorporated herein by reference.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

There are a number of critical knowledge gaps in relation to their screening and treatment, e.g., with illudin-based treatments. There is insufficient knowledge of patient characteristics, including genetic profiles, for optimal stratification of patients into response groups at the time of diagnosis. There is also insufficient knowledge of the risk factors for developing or dying from these cancers and a lack of effective implementation of evidence into clinical practice. This lack of knowledge means that predicting which patients will have the best outcomes with specific treatments is suboptimal.

Accordingly, there is a need for improved methods for assessing and determining a patient's sensitivity to an anticancer agent.

### BRIEF SUMMARY

Provided herein are methods for determining sensitivity of a cancer to an anti-cancer agent, comprising determining an expression level of at least one biomarker, an expression level of at least one gene associated with DNA repair, a transcription level of at least one thereof, or any combination thereof. Further provided herein are methods, wherein a reduced expression or transcription level compared to a standard or control sample indicates a sensitivity of the cancer to the anti-cancer agent. Further provided herein are methods, wherein the anti-cancer agent comprises illudin or an illudin analog. Further provided herein are methods, wherein the cancer comprises a solid tumor. Further provided herein are methods, wherein the solid tumor is a tumor of the breast, central nervous system, colon, skin, lung, ovary, prostate, or kidney. Further provided herein are methods, wherein the cancer is a breast cancer, a central nervous system cancer, a colon cancer, a melanoma, a lung cancer, an ovarian cancer, a prostate cancer, or a renal cancer. Further provided herein are methods, wherein the at least one gene associated with DNA repair is a DNA Damage Repair gene (DDRG). Further provided herein are methods, wherein the at least one gene associated with DNA repair is a Nucleotide Excision Repair (NER) gene. Further provided herein are methods, wherein the at least one gene associated with DNA repair is a homologous recombination (HR) gene. Further provided herein are methods, wherein the at least one gene associated with DNA repair comprises BRCA1, BRCA2, ATM, ATR, ERCC2, ERCC3, ERCC4, ERCC5, ERCC6, FANCD2, RAD51, or PALB2. Further provided herein are methods, wherein the at least one biomarker comprises expression of RPA1, FANCE, FANCL, ERCC8/CSA, BRIP1, FANCF, MRE11A, BLM, ERCC3/XPB, FANCM, FANCB, FANCE, CHEK1, FANCI, ATM, ERCC4/XPG, ERCC6/CSB, CHEK2, ERCC2/XPD, OR ERCC3 genes. Further provided herein are methods, wherein the at least one biomarker comprises expression of RPA1, FANCE, FANCL, BRIP1, FANCF, MRE11A, BLM, CHEK1, CHEK2, or ATM. Further provided herein are methods, wherein the at least one biomarker comprises expression of PTGRI OR SDC4. Further provided herein are methods, wherein the transcription level is determined by measuring an amount of mRNA transcribed from the at least one gene associated with DNA repair.

Provided herein are methods of screening an anti-cancer agent for use in treatment of a cancer in a subject in need thereof comprising determining a change in an expression level of at least one biomarker, an expression level of at least one gene associated with DNA repair, a transcription level of at least one thereof, or any combination thereof in a sample from the subject following exposure to the anti-cancer agent. Further provided herein are methods, wherein a decrease in the expression or transcription level indicates a sensitivity to the anti-cancer agent. Further provided herein are methods, wherein the anti-cancer agent comprises illudin or an illudin analog. Further provided herein are methods, wherein the cancer comprises a solid tumor. Further provided herein are methods, wherein the solid tumor is a tumor of the breast, central nervous system, colon, skin, lung, ovary, prostate, or kidney. Further provided herein are methods, wherein the cancer is a breast cancer, a central nervous system cancer, a colon cancer, a melanoma, a lung cancer, an ovarian cancer, a prostate cancer, or a renal cancer. Further provided herein are methods, wherein the at least one gene associated with DNA repair is a DNA Damage Repair gene (DDRG). Further provided herein are methods, wherein the at least one gene associated with DNA repair is a Nucleotide Excision Repair (NER) gene. Further provided herein are methods, wherein the at least one gene associated with DNA repair is a homologous recombination (HR) gene. Further provided herein are methods, wherein the at least one gene associated with DNA repair comprises BRCA1, BRCA2, ATM, ATR, ERCC2, ERCC3, ERCC4, ERCC5, ERCC6, FANCD2, RAD51, or PALB2. Further provided herein are methods, wherein the at least one biomarker comprises expression of RPA1, FANCE, FANCL, ERCC8/CSA, BRIP1, FANCF, MRE11A, BLM, ERCC3/XPB, FANCM, FANCB, FANCE, CHEK1, FANCI, ATM, ERCC4/XPG, ERCC6/CSB, CHEK2, ERCC2/XPD, OR ERCC3 genes. Further provided herein are methods, wherein the at least one biomarker comprises expression of RPA1, FANCE, FANCL, BRIP1, FANCF, MRE11A, BLM, CHEK1, CHEK2, or ATM. Further provided herein are methods, wherein the at least one biomarker comprises expression of PTGRI OR SDC4. Further provided herein are methods, wherein the transcription level is determined by measuring an amount of mRNA transcribed from the at least one gene associated with DNA repair.

Provided herein are methods for treating a cancer in a subject in need thereof, comprising administering to the subject an effective amount of an anti-cancer agent, wherein the subject has a reduced expression level of a biomarker, a reduced expression of at least one gene associated with DNA repair, a reduced transcription level of at least one thereof, or any combination thereof. Further provided herein are methods, wherein the reduced expression or transcription level is compared to a standard or control sample. Further provided herein are methods, wherein the anti-cancer agent comprises illudin or an illudin analog. Further provided herein are methods, wherein the cancer comprises a solid tumor. Further provided herein are methods, wherein the solid tumor is a tumor of the breast, central nervous system, colon, skin, lung, ovary, prostate, or kidney. Further provided herein are methods, wherein the cancer is a breast cancer, a central nervous system cancer, a colon cancer, a melanoma, a lung cancer, an ovarian cancer, a prostate cancer, or a renal cancer. Further provided herein are methods, wherein the at least one gene associated with DNA repair is a DNA Damage Repair gene (DDRG). Further provided herein are methods, wherein the at least one gene associated with DNA repair is a Nucleotide Excision Repair (NER) gene. Further provided herein are methods, wherein the at least one gene associated with DNA repair is a homologous recombination (HR) gene. Further provided herein are methods, wherein the at least one gene associated with DNA repair comprises BRCA1, BRCA2, ATM, ATR, ERCC2, ERCC3, ERCC4, ERCC5, ERCC6, FANCD2, RAD51, or PALB2. Further provided herein are methods, wherein the at least one biomarker comprises expression of RPA1, FANCE, FANCL, ERCC8/CSA, BRIP1, FANCF, MRE11A, BLM, ERCC3/XPB, FANCM, FANCB, FANCE, CHEK1, FANCI, ATM, ERCC4/XPG, ERCC6/CSB, CHEK2, ERCC2/XPD, OR ERCC3 genes. Further provided herein are methods, wherein the at least one biomarker comprises expression of RPA1, FANCE, FANCL, BRIP1, FANCF, MRE11A, BLM, CHEK1, CHEK2, or ATM. Further provided herein are methods, wherein the at least one biomarker comprises expression of PTGRI OR SDC4. Further provided herein are methods, wherein the transcription level is determined by measuring an amount of mRNA transcribed from the at least one gene associated with DNA repair.

Provided herein are kits, for use in determining sensitivity of a specimen to an anti-cancer agent according to any of the method described herein, wherein the kit comprises one or more reagents, standards, and instructions for use thereof, wherein the standards comprise one or more biomarkers or expression or transcription products, providing a threshold level, or a target level for screening sensitivity of the specimen to the anti-cancer agent.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a line graph of in vitro concentration of LP-184 in plasma over time.

FIG. 1B is a line graph of in vivo pharmacokinetics of LP-184.

FIG. 2 is a bar graph showing Log₁₀ of LP-184 IC50 in a range of cancer cell lines.

FIG. 3 is a bar graph showing LP-184 IC50 in pancreatic cell lien Panc03.27 before and after ERCC4 depletion.

FIGS. 4A and 4B are line graphs showing sensitivity of PC3M cells with and without BRCA2 depletion, after treatment with LP-184 and Olaparib.

FIG. 5A is images of LuCaP 96 organoids, stained to show live or dead cells, showing dose-dependent cell death after treatment with LP-184.

FIG. 5B is a line graph depicting the mean organoid number/field vs. dose LP-184.

FIG. 5C is a bar chart showing dead cells/field vs. dose LP-184.

FIG. 6 is a t-SNE chart of expression of NER genes as a predictor of LP-184 sensitivity.

FIG. 7 is scatter plot of predicted IC50 values of LP-184 in cells with high or low expression of NER genes ERCC3 and ERCC6.

### DETAILED DESCRIPTION

One aspect of this application includes the anti-cancer agent sensitivity determination markers, which includes the negative correlation with biomarkers or DNA Damage Repair Genes or DDRG transcription levels (that is tumors that have reduced expression of DDRG genes). The DDRG transcript levels correlated negatively with sensitivity to illudin-based treatments or correlated with true responders to illudin-based treatments. That is, solid tumors with mutations of DDRG are more sensitive to illudin-based treatments. The use of this marker, alone or with others, can improve the gaps in treatment of cancers (particularly, solid tumors) by biomarker-based screening tests enabling precision medicine-based therapies to patients.

Another aspect includes a method of treating cancer in a subject in need thereof. The method comprising administering to the subject an effective amount of an illudin or illudin analog, wherein the subject has a defect in DNA repair or a defect in NER or HR gene pathways. One embodiment includes a method for determining sensitivity of a subject having cancer to an illudin-based anti-cancer agent. The method includes measuring a level or presence of various DDRG genes or transcript level in the specimen from the subject. The level or the absence of these genes indicates subject sensitivity to the illudin-based anti-cancer agent. In specific embodiments, the level of expression of the plurality of biomarkers of sensitivity is determined by detecting the level of mRNA transcribed from genes encoding the plurality of biomarkers of sensitivity.

In another embodiment, includes a method for determining sensitivity of a subject having cancer to an illudin-based anti-cancer agent using NER and/or HR gene expression. In one example, that is, certain pancreatic tumors that harbored genetic alternations in the following DNA repair pathways—NER (nucleotide excision repair) and HR (homologous recombination)—had increased sensitivity, by 2-fold, to LP-184. Other cancers that have DNA repair pathway mutations include mutations or deficiencies in genes such as: BRCA1, BRCA2, ATM, ATR, ERCC2, ERCC3, ERCC4, ERCC5, ERCC6, FANCD2, RAD51 and PALB2.

In another embodiment, one or more of the following gene markers, that is the reduced expression or transcription of these markers, indicated more sensitivity to illudin or LP-184. These markers include the genes below in Table 1.

TABLE 1
DDRG

ATM
BLM
BRIP1
CHEK1
CHEK2
ERCC2/XPD
ERCC3
ERCC3/XPB
ERCC5/XPG
ERCC6/CSB
ERCC8/CSA
FANCB
FANCE
FANCF
FANCI
FANCL
FANCM
MRE11A
RPA1

Among the several high-ranking genes that negatively correlate significantly with sensitivity to LP184, several DDRGs (RPA1, FANCE, FANCL, BRIP1, FANCF, MRE11A, BLM, CHEK1, CHEK2, ATM, as listed in Table 1) can result in improved illudin or LP184 sensitivity.

In yet another embodiment, the two markers PTGRI and SDC4 may be used as the markers to indicate subject sensitivity to an illudin-based anti-cancer agent/treatment.

As used herein, the term “illudin” includes analogs and the compounds with formula I:

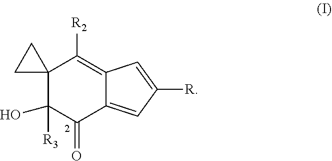

The R1, R2 and R3 are independently (C1-C4) alkyl, methyl, or hydroxyl. The term illudin may include HydroxyMethylAcylfulvene (Irofulven), which has the following formula II:

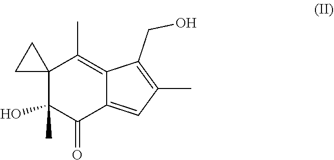 [SMILES: CC1=C(CO)C2=C(C)C3(CC3)[C@@](C)(O)C(=O)C2=C1]

The term illudin also may include HydroxyUreaMethylAcylfulvene, (LP184), which has the following formula III

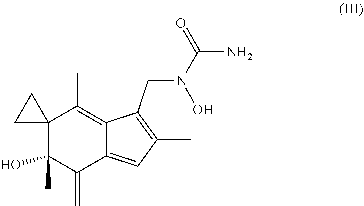 [SMILES: C=C1C2=CC(C)=C(CN(O)C(N)=O)C2=C(C)C2(CC2)[C@@]1(C)O]

In one example, the illudin includes the analog Irofulven.

In a case where a combination of genes is used, screening of an anti-cancer agent sensitivity enhancer can be performed through employment, as an index, of variation in expression of a DDRG gene combination of genes after exposure to the illudin-based anti-cancer agent. That is, a cancer with decreased DDRG transcript levels determines sensitivity to the anti-cancer agent.

In another case where a combination of genes is used, screening of an anti-cancer agent sensitivity enhancer can be performed through employment, as an index, of variation in expression of a DDRG gene combination of genes after exposure to the illudin-based anti-cancer agent. The DDRG or DNA Damage Repair Genes include BRCA1, BRCA2, ATM, ATR, ERCC2, ERCC3, ERCC4, ERCC5, ERCC6, FANCD2, RAD51 and PALB2.

In order to carry out the method of the present invention for determining sensitivity of a specimen to an anti-cancer agent, preferably, a kit containing a protocol for measuring any of the substances present in the specimen is employed. The kit contains reagents for measuring any of these substances, an indication of an instruction manual for use of the reagent, standards for determining the presence or absence of sensitivity to the illudin-based anti-cancer agent, etc. The standards include (relative) standard levels of these markers, a (relative) high threshold level, a (relative) low threshold level, factors affecting the measurements, the degree of the effects, etc. These substance levels may be set so as to suit the illudin-based anti-cancer agent selected. The sensitivity determination may be performed in the same manner on the basis of the standards.

Screening of an illudin-based anti-cancer agent can be performed by means of the illudin-based anti-cancer agent sensitivity determination markers as an index.

That is, a substance which can vary the level of the anti-cancer agent sensitivity determination markers in vitro or in vivo is evaluated as an anti-cancer agent. For example, in an in vitro case, a substance which varies the anti-cancer agent sensitivity determination marker level in various cancer cells after exposure to the substance can serve as an anti-cancer agent. Also, when the anti-cancer agent sensitivity determination marker level in a cancer-bearing animal is varied after administration of a substance thereto, the substance can serve as an anti-cancer agent. If the anti-cancer agent is expected to exhibit a pharmacological effect, the increase in anti-cancer agent sensitivity determination markers level is observed before occurrence of tumor shrinkage or attaining cytocidal effect. Therefore, screening based on the anti-cancer agent sensitivity determination marker levels as an index can realize, for a shorter period of time, determination whether or not the test substance serves as a useful anti-cancer agent, whereby efforts and cost involved in the development of anti-cancer agents are greatly expected to be reduced or at least personalized.

When the cancer has no sensitivity to an anti-cancer agent, no or less pharmacological effect can be expected from the anti-cancer agent. If such a pharmaceutically impotent anti-cancer agent is continuously administered to the patient, the cancer may progress, and side effects may be aggravated. Thus, the anti-cancer agent sensitivity determination markers may be employed not only to determine therapeutic response to the anti-cancer agent, but also to greatly contribute to prevention of aggravation of side effects, which would otherwise be caused by continuous administration of a pharmaceutically impotent anti-cancer agent.

A reference value is determined for each biomarker. Typically, the reference value can be a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). A person skilled in the art may compare the biomarkers expression levels (obtained according to the method of the invention with a defined threshold value). In one embodiment of the present invention, the threshold value is derived from the biomarkers expression level (or ratio, or score) determined in a blood sample derived from one or more subjects who are responders to gene therapy and gemcitabine combination treatment. In one embodiment of the present invention, the threshold value may also be derived from biomarker expression level (or ratio, or score) determined in a blood sample derived from one or more subjects who are non-responders to gene therapy and gemcitabine combination treatment. Furthermore, retrospective measurement of the biomarker expression levels (or ratio, or scores) in properly banked historical subject samples may be used in establishing these threshold values.

The terms “sensitivity,” “responsive” and “responsiveness,” as used herein, refer to the likelihood that a cancer treatment (e.g., LP184) has (e.g., induces) a desired effect, or, alternatively, refer to the strength of a desired effect caused or induced by the treatment in a cell (e.g., a cancer cell), a tissue (e.g., a tumor), or a patient having cancer (e.g., a human having cancer). For example, the desired effect can include inhibition of the growth of a cancer cell in vitro by more than 10%, 20%, 30%, 40%, 50%, 60%, 70%, 80%, 90%, or 100% relative to the growth of a cancer cell not exposed to the treatment. The desired effect can also include reduction in tumor mass by, e.g., about 10%, 20%, 30%, 40%, 50%, 60%, 70%, 80%, 90%, or 100%. Sensitivity to treatment may be determined by a cell proliferation assay, e.g., a cell-based assay, which measures the growth of treated cells as a function of the absorbance of the cells of an incident light beam, such as the NCI60 assays described herein. In this assay, lesser absorbance indicates lesser cell growth, and thus, sensitivity to the treatment. A greater reduction in growth indicates more sensitivity to the treatment.

Through employment, in combination, of the biomarker analysis and a therapy with illudin-based anti-cancer agent, the cancer treatment can be drastically enhanced or personalized to a subject or patient. DDR-deficient tumors are sensitive to illudins and LP184, which can be a treatment for solid tumors with reduced or no DDRG transcript levels. Therapy can include radiation or other therapies.

### EXAMPLES

### Example 1: Plasma Stability and Pharmacokinetic Profile of LP-184

1 mg/mL LP-184 was administered as an intravenous bolus dose to CD-1 mice. Samples were collected from 3 mice per time point and 9 time points including pre-dose were reported.

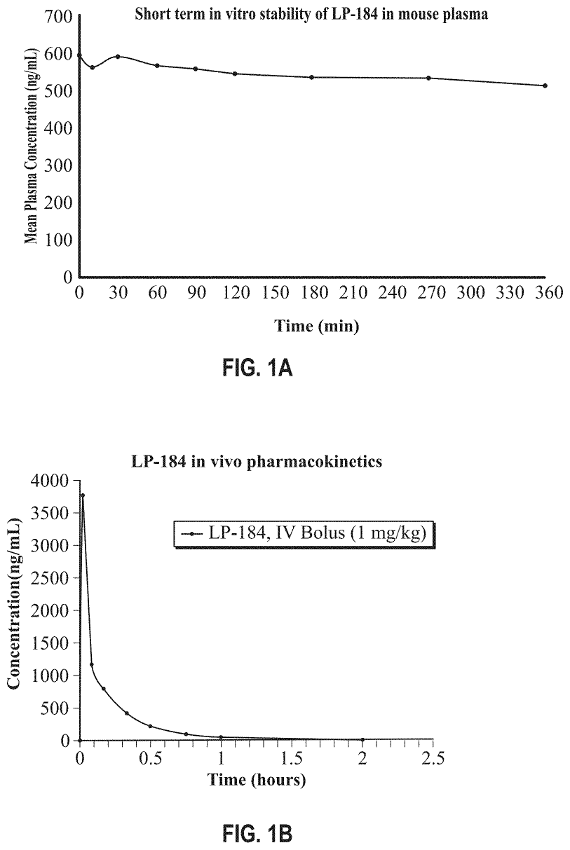

Plasma stability of LP-184 was assessed in vitro over 2.5 hours. As shown in FIG. 1A, LP-184 had a favorable in vitro plasma stability, with plasma concentration maintained to at least 360 minutes at 25° C. (benchtop).

In vivo pharmacokinetic data was plotted and a plasma half-life of 16.2 minutes was calculated (see, FIG. 1B). Calculated pharmacokinetics are shown in Table 2.

TABLE 2

| Pharmacokinetic parameter | LP-184 |
|---|---|
| Half life (min) | 16.2 |
| AUC (h*ng/mL) | 517 |
| Cₘₐₓ (ng/ml) | 3760 |

### Example 2: IC50 of LP-184 in Spectrum of Tumor Cell Lines

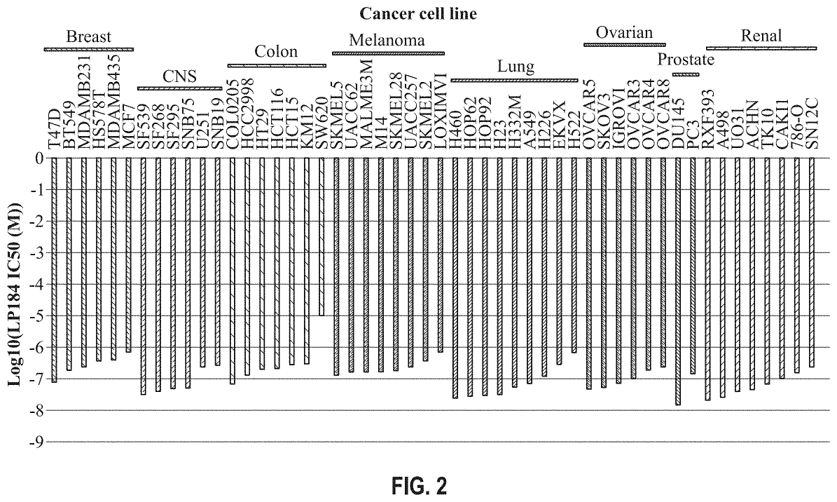

A spectrum of tumor cell lines were screened for LP-184 efficacy. FIG. 2 shows Log10 IC50 of the 52 solid tumor cell lines tested. Prostate cancer cell line DU145 was the most sensitive to LP-184 among the tested cell lines.

### Example 3: Pre-seeding and Seeding Assay design

Pre-seeding assay and QA/QC

Pre-Assay Day 0: Tissue was minced and gently dissociated as described in Example 4. Cells were cultured overnight in ultra-low attachment (ULA) dishes.

Pre-Assay Day 1: Cultures were assessed for contamination (QC1). Cultures were then filtered through 500 μm and 200 μm filters. Live cells were quantified using CellTiter-Glo (CTG) standard curve vs. PDX models. Cell Titer-Glo assay was performed as a Pass/Fail checkpoint (QC2). Additional vials of any models that failed QC2 were thawed, dissociated, and cultured overnight. Models that passed QC2 remained in culture in ULA dishes.

Pre-Assay Day 2 and 3: Day 1 steps were repeated with any newly dissociated model cultures. If any models failed QC2 for a third round, the model was considered non-viable and a replacement model was used in its place.

Post-Seeding Assay and QA/QC

Assay Day 0: Live cells were quantified using CTG standard curve vs. PDX models. Model viability was verified to confirm sufficient to seed full-scale assay (QC3). Seeding density was normalized based on CTG and tumor fragments were seeded in assay plates. Fragments were allowed to adhere to Cell-Tak coated plates for 1 hour. Test agents were applied, and “baseline” plates were processed for CTG and imaging.

Assay Day 5 (endpoint): CellTiter-Glo data was gathered for assay plates treated with full test agent dose response. As a CellTiter-Glo pass/fail checkpoint, positive control values must be significantly lower than vehicle control values (QC4). Assay plates treated with a subset of test agent doses were labeled and imaged. “Baseline” plates were processed for CTG and imaging.

Data Analysis

To remove outliers, prior to fitting a dose response curve, data samples that differed by more than 2 standard deviations from both the mean of the data across the plate and from the mean of the data within the relevant treatment condition were removed automatically. Outliers were removed only if at least n=4 data replicated would remain. Where deemed appropriate, anomalous data samples were removed manually. To complete the regression analysis, dose response curves were plotted and EC50 values were reported when the R2 value of the fit was at least 0.65.

Strictly standardized mean difference (SSMD) was assessed for data points;

when SSMD≥1.5 but SSMD <20, the SSMD value is printed about the data point;

when SSMD≥2 the SSMD value is printed in bold typeface;

when SSMD L 20, its value is printed as “>20” or “<20” as appropriate.

Results of CellTiter-Glo data was reported as % viability normalized to a negative (vehicle) control group. Representative images from a live-dye/antibody palette are included for: negative controls, positive controls, and 2 doses of test agent. The live-dye palette consisted of: Hoechst (all nuclei), CellTracker Green (live cell dye), p-γH2A.X (DNA damage marker), EdU (incorporation indicates S-phase).

### Example 4: Tumor Dissociation, Seeding, and Treatment

Cryopreserved tumors were thawed and manually dissociated in to 2 mm pieces. After manual dissociation, the tumors were further dissociated via a Miltenyi GentleMACs system (Miltenyi Biotec, Auburn, CA). Dissociated tumor fragments were cultured overnight (and up to 7 days) in ultra-low attachment plates. Tumor fragments were filtered through 500 μm or 200 μm filters before viability was assessed via CellTiter-Glo. Tumor fragments were seeded into Cell-Tak coated, 384-well low volume COC plates and allowed 1 hour to attach to the coated surface. After attachment, the tumor fragments were treated with negative controls, positive controls, and test agents. Negative controls comprised of 0.1% DMSO for DMSO-soluble test agents, and PBS for aqueous test agents. Positive control comprise 10% DMSO in complete medium. Plates were then incubated at 37° C. and 5% CO₂ for the duration of the assay.

### Example 5: Imaging Tumor Fragments Labeled with Live-Dye/Antibody Palette

Live tumor fragments were pulsed for 3 hours with EdU and 1 hour with CellTracker Green CMFDA before fixation with 4% PFA in PBS. EdU is incorporated into DNA as a marker of S-phase growth. CellTracker Green CMFDA is a live cell dye. After fixation, EdU signal is developed according to manufacturer's instructions using the Thermo Fisher Click-IT EdU Alexa Fluor 647 HCS Assay (Thermo Fisher Scientific, Waltham, MA). CellTracker- and EdU-labeled plates were then labeled with antibody via standard immunofluorescent protocols. Lastly, tumor fragments were labeled with Hoechst pan nuclear marker. Plates were imaged using either a Thermo Fisher CX7-LED HCS or CX7-LZR HCS platform.

### Example 6: LP-184 Sensitivity Correlates Negatively with Transcript Levels of NER Pathway Genes

A 15-20% subset of pancreatic cancer or tumors carry mutations in DNA repair pathway (BRCA1/BRCA2/PALB2/RAD51/ATM/FANCD2). Additionally, mutations in nucleotide excision repair (NER) genes (ERCC2/3/4/5/6) have been reported in −5% of PDAC. LP-184 is a novel synthetic small molecule acylfulvene analog. Once activated by PTGR1, highly reactive LP-184 nucleophile creates covalent DNA adducts that are selectively repaired via Nucleotide Excision Repair (NER) mechanism coupled to transcription (TC-NER) and/or homologous recombination (HR). Mutation or expression driven TC-NER and HR deficiency would predispose PDAC cells to increased sensitivity to LP-184.

Results: LP-184 activity in DNA repair-deficient tumors. LP-184 chemosensitivity in genetically defined PDAC models in vitro, ex vivo, and in xenografts. Testing in six different pancreatic cancer cell lines (Capan-1, CFPAC-1, Panc1, MiaPaCa2, Panc03.27 and BxPC-3) resulted in very potent inhibition with LP-184 IC50 values ranging from 114 to 182 nM. In this cell line panel, LP-184 sensitivity correlated negatively with transcript levels of an NER pathway gene ERCC8 (r=−0.94). In comparison to these PDAC cell lines, a normal pancreatic epithelial cell line HPNE was 3-6 times less sensitive to LP-184 (IC50 670 nM). Ex vivo cultures of 4 out of 5 low-passage patient-derived xenografts with HR deficiency showed nanomolar sensitivity to LP-184 with IC50s ranging from 45 to 270 nM. These tumor graft models which were at least 6 times less sensitive to olaparib in the same assay. Depletion of ERCC4 enhanced sensitivity to LP-184 about 2-fold relative to the parental cell line.

To define PTGR1 as a biomarker for LP-184 activity, CRISPR/Cas9-mediated gene editing depleted PTGR1 expression. PTGR1-null Capan-1 cell line-derived xenografts were poorly sensitive to LP184, whereas PTGR1-expressing xenografts showed near complete tumor regression in all LP184 treated animals with 109% tumor growth inhibition relative to the control group in this study. Furthermore, PTGR1 depleted cells were completely resistant to LP184 in vitro.

Summary. The data demonstrates that PDAC models carrying a range of DNA repair pathway mutations are highly sensitive to LP-184 in vitro and in vivo. Increased PTGR1 expression is a validated biomarker for LP184 cytotoxicity, and is the exclusive convertase of LP184 to an active alkylator drug.

Table 3 shows gene correlations and drug sensitivity.

TABLE 3

| | | LP-184 | Max | Olaparib | Max | |
|---|---|---|---|---|---|---|
| | Tumor | IC50 | inhibition | IC50 | inhibition | DDR Gene |
| Model | type | [nM] | (%) | [nM] | (%) | Mutations |
| CTG-0166 | NSCLC | 57 | 97 | 720 | 77 | ATM, FANCD2, NBN |
| CTG-1194 | NSCLC | 31 | 91 | NR | 52 | ATM |
| CTG-2532 | NSCLC | 54 | 99 | 17000 | 81 | CHEK1, FANCA, |
| | | | | | | NBN, RAD50 |
| CTG-0302 | Pancreatic | 110 | 91 | NR | 46 | BRCA2, ATM, BLM, |
| | | | | | | FANCA |
| CTG-0314 | Pancreatic | 27 | 82 | 1700 | 80 | BRCA2, CDK12, |
| | | | | | | PALB2 |
| CTG-0381 | Pancreatic | 2900 | 94 | QC fail | QC fail | BRCA1, BRCA2, ATM |
| CTG-1522 | Pancreatic | 45 | 97 | 7900 | 81 | ATR, BRIP1, PARP1 |
| CTG-1643 | Pancreatic | 57 | 77 | NR | 65 | BRCA1, BRIP1, |
| CTG-2429 | Prostate | 92 | 92 | 18000 | 68 | ATM, ATR, PALB2, |
| CTG-2440 | Prostate | 31 | 95 | NR | 59 | PMS2 |
| CTG-3167 | Prostate | 54 | 97 | 4200 | 48 | BRCA2, ATM, |
| | | | | | | FANCA, FANCI, |
| | | | | | | FANCM |
| CTG-3537 | Prostate | 54 | 98 | NR | 29 | BRCA2, CDK12, |
| | | | | | | FANCI, RAD54L, |

The above table was obtained used the basic steps shown attached method.

### Example 7: LP-184 as Lethal Agent in Treatment of Tumors with Specific DDR Deficiencies

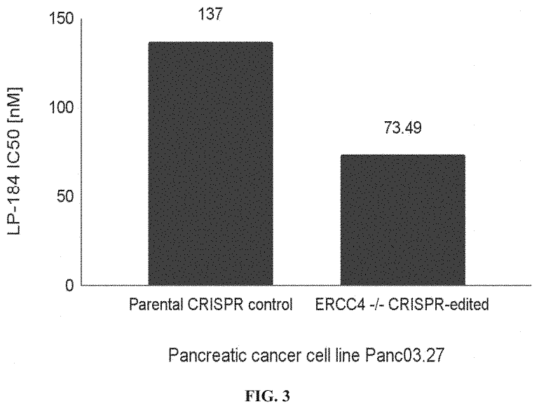

To demonstrate enhancement of LP-184 sensitivity, ERCC4, a transcription coupled nucleotide excision repair/TC-NER component, was suppressed using standard CRISPR techniques in pancreatic cancer Panc03.27 cells. As shown in FIG. 3, suppression of ERCC4 enhances LP-184 sensitivity approximately 2-fold relative to isogenic parental CRISPR control.

LP-184 has a potential to be a synthetic lethal agent in treatment of tumors with specific DNA damage repair (DDR) deficiencies.

### Example 8: LP-184 Potency in Prostate Cancer Cells with DDR Mutations

LP-184 shows nanomolar in vitro potency in prostate cancer cell lines harboring damaging DDR mutations.

Three prostate cancer cell lines were induced with damaging DDR gene mutations using standard CRISPR techniques. Cell line 22RV1 was induced with a BRCA2 mutation. DU145 cells were induced with ERCC6 and FANCI mutations. LNCAP cells were induced with ATM and ERCC3 mutations. Table 4 shows nanomolar in vitro potency in prostate cancer cell lines harboring damaging DDR mutations.

TABLE 4

| Prostate Cancer | Damaging DDR | LP-184 |
|---|---|---|
| Cell line | gene mutations | IC50 (nM) |
| 22RV1 | BRCA2 | 20.5 |
| DU145 | ERCC6, FANCI | 44.9 |
| LNCAP | ATM, ERCC3 | 219 |

### Example 9: Increased Sensitivity to LP-184 in BRCA2-Depleted Cancer Cells

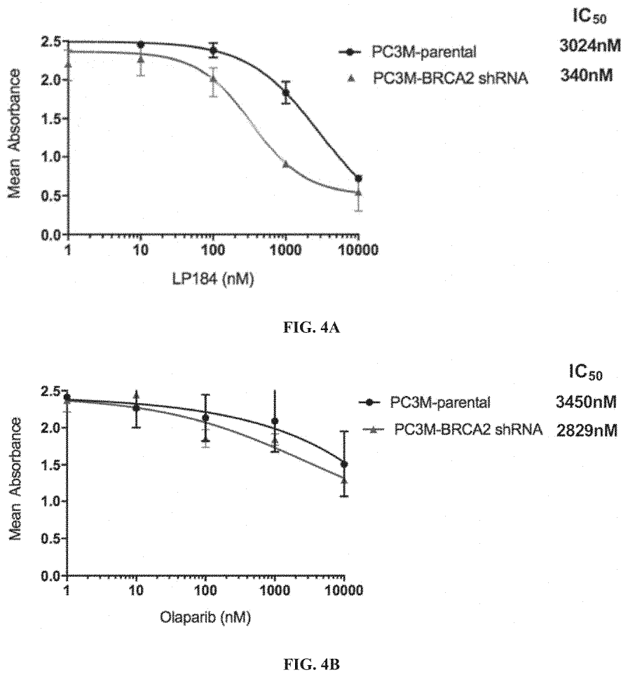

BRCA2-depleted PC3M human prostate cancer cells were treated with LP-184 and PARP inhibitor olaparib (Lynparza®). FIG. 4A shows suppression of growth in PC3M (BRCA2-) cells compared to parental cells, after treatment with LP-184. A calculated IC50 of 3024 nM in patental PC3M cells is reduced 9-fold, to 340 nM in cells after depletion of BRCA2—. In contrast, FIG. 4B shows no significant change in sensitivity to treatment with PARP inhibitor olaparib in PC3M cells after BRCA2 depletion.

Comparison of IC50 between BRCA2-depleted cells treated with olaparib or LP184 indicates an 8-fold increased sensitivity to LP-184 over Olaparib.

### Example 10: Dose-Dependent Cell Killing in Cancer Cells with DDR Mutations

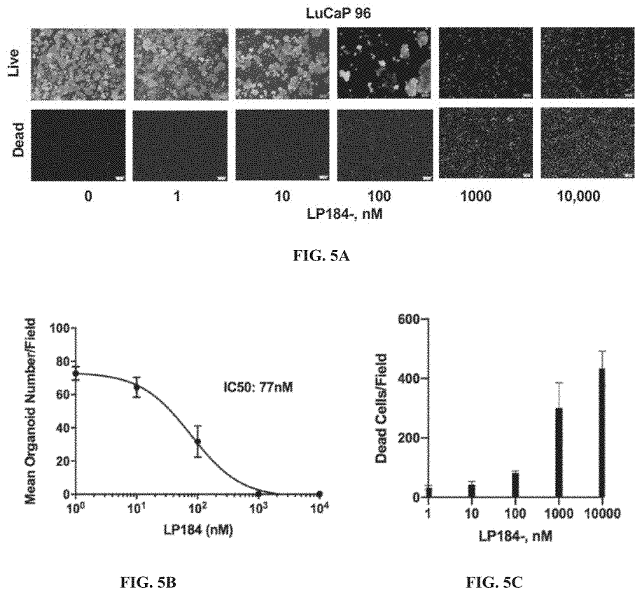

LuCaP96 cells, a prostate cancer PDX model with inactivating BRCA2 and CHEK2 mutations, were proliferated as organoids. Staining of live and dead cells in culture is shown in FIG. 5A, indicating a dose-dependent shift from live to dead cells. FIG. 5B shows quantification of organoids at each treatment dose, indicating an IC50 of 77 nM. A corresponding quantification of dead cells at each treatment dose shown in FIG. 5C shows increasing dead cells with higher dose. Treatment with LP-184 shows dose-dependent cell kill in the nanomolar range in the prostate cancer organoid model.

### Example 11: Prediction of LP-184 Sensitivity Based on NER Gene Expression

The expression level of the following NER genes were able to classify the top 15 predicted sensitive and top 15 predicted insensitive Glioblastoma (GBM) tumor sample records into distinct sensitivity groups out of a total of 166 records from TCGA (The Cancer Genome Atlas), whereas parameters such as gender and race could not classify the samples. The genes include: POLE, RFC3, POLE, LIG1, PARP2, RFC5, LIG3, RFC4, INO80B, POUCH, PCNA, ACTR5, POLD1, GPS1, NFRKB, XAB2, POLRD2, POLD3, IN-0801H, ZNF830, ERCC8, POLE3, RPA2, GTF2H4, PARP1, ERCC3, POLR2B, RUVBL1, MCRS1, USP45, COPS5, POLR2I, COPS3, UBE2N, INO80D, PRPF19, COPS7B, XRCC1, ERCC6, UBE2I, POLR2E, CCNH, ACTR8, USP7, POLR2G, UBB, ERCC5, INO80C, RPA1, POLR2J, UBE2V2, POLR2H, RPA3, AQR, RFC1, PPIE, POLD2, ERCC2, POLE4, SUMO4, COPS4, YY1, RAD23 A, COPS6, XPA, MNAT1, POLR2K, RAD23B, CUL4B, POLR2A, PIAS1, ISY1, GTF2H5, POLK, EP300, UBA52, XPC, ERCC1, RBX1, RNF111, TCEA1, GTF2H3, CUL4A, INO80, COPS7A, COPS8, RPS27A, PIAS3, TFPT, GTF2H2, CETN2, SUMO3, CHD1L, GTF2H1, COPS2, UBC, CDK7. ACTB, POLR2L, ELL, DDB2, POLD4

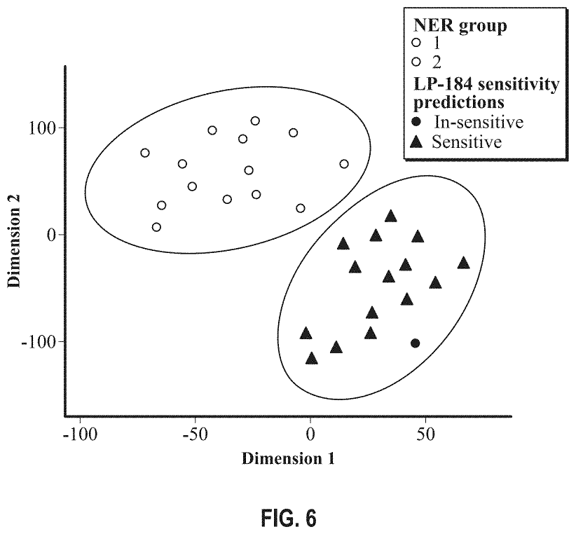

GBM patient expression data was plotted using t-SNE analysis. t-SNE clusters identified subgroups with high and low expression of NER genes which had distinct predicted LP-184 sensitivity. FIG. 6 shows 15/16 GBM samples from NER group 1 were predicted to be sensitive to LP-184.

### Example 12: Predicted LP-184 Sensitivity in GBM with Low ERCC3/6 Expression

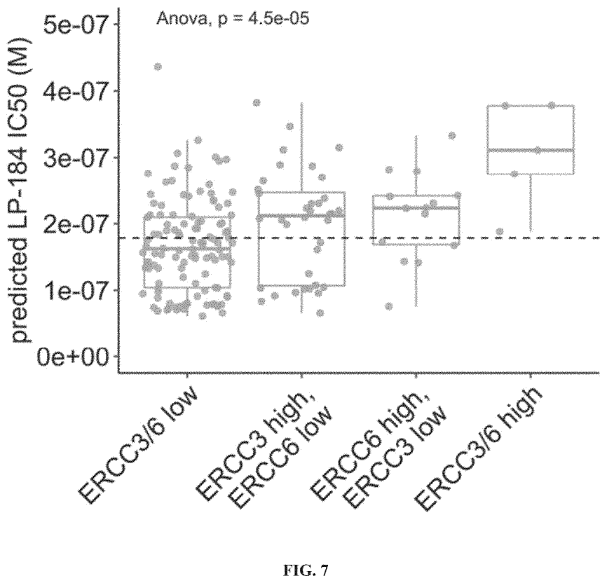

TCGA data from clinical GBMs with assessed expression of the NER genes ERCC3/6 were plotted for predicted LP-184 sensitivity (N=173). FIG. 7 shows GBMs with low expression of the NER genes ERCC3/6 were predicted to be more sensitive to LP-184 relative to those with high expression across evaluable GBM TCGA records.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for determining sensitivity of a cancer to an anti-cancer agent, comprising determining an expression level of at least one biomarker, an expression level of at least one gene associated with DNA repair, a transcription level of at least one thereof, or any combination thereof, wherein the anti-cancer agent comprises illudin or an illudin analog.

2. The method of claim 1, wherein the illudin analog is HydroxyUreaMethylAcylfulvene.

3. The method of claim 1, wherein the illudin analog is Irofulven.

4. The method of claim 1, wherein a reduced expression or transcription level compared to a standard or control sample indicates a sensitivity of the cancer to the anti-cancer agent.

5. The method of claim 1, wherein the cancer comprises a solid tumor.

6. The method of claim 5, wherein the solid tumor is a tumor of the breast, central nervous system, colon, skin, lung, ovary, prostate, or kidney.

7. The method of claim 1, wherein the cancer is a breast cancer, a central nervous system cancer, a colon cancer, a melanoma, a lung cancer, an ovarian cancer, a prostate cancer, or a renal cancer.

8. The method of claim 1, wherein the at least one gene associated with DNA repair is a DNA Damage Repair gene (DDRG).

9. The method of claim 1, wherein the at least one gene associated with DNA repair is a Nucleotide Excision Repair (NER) gene.

10. The method of claim 1, wherein the at least one gene associated with DNA repair is a homologous recombination (HR) gene.

11. The method of claim 1, wherein the at least one gene associated with DNA repair comprises BRCA1, BRCA2, ATM, ATR, ERCC2, ERCC3, ERCC4, ERCC5, ERCC6, FANCD2, RAD51, or PALB2.

12. The method of claim 1, wherein the at least one biomarker comprises expression of RPA1, FANCE, FANCL, ERCC8/CSA, BRIP1, FANCF, MRE11A, BLM, ERCC3/XPB, FANCM, FANCB, FANCE, CHEK1, FANCI, ATM, ERCC4/XPG, ERCC6/CSB, CHEK2, ERCC2/XPD, OR ERCC3 genes.

13. The method of claim 1, wherein the at least one biomarker comprises expression of RPA1, FANCE, FANCL, BRIP1, FANCF, MRE11A, BLM, CHEK1, CHEK2, or ATM.

14. The method of claim 1, wherein the at least one biomarker comprises expression of PTGRI OR SDC4.

15. The method of claim 1, wherein the transcription level is determined by measuring an amount of mRNA transcribed from the at least one gene associated with DNA repair.

16. A method of screening an anti-cancer agent for use in treatment of a cancer in a subject in need thereof comprising determining a change in an expression level of at least one biomarker, an expression level of at least one gene associated with DNA repair, a transcription level of at least one thereof, or any combination thereof in a sample from the subject following exposure to the anti-cancer agent, wherein the anti-cancer agent comprises illudin or an illudin analog.

17. The method of claim 16, wherein a decrease in the expression or transcription level indicates a sensitivity to the anti-cancer agent.

18. The method of claim 16, wherein the anti-cancer agent is HydroxyUreaMethylAcylfulvene.

19. The method of claim 16, wherein the anti-cancer agent is Irofulven.

20. The method of claim 16, wherein the cancer comprises a solid tumor.

21. The method of claim 20, wherein the solid tumor is a tumor of the breast, central nervous system, colon, skin, lung, ovary, prostate, or kidney.

22. The method of claim 16, wherein the at least one gene associated with DNA repair is a DNA Damage Repair gene (DDRG).

23. The method of claim 16, wherein the at least one gene associated with DNA repair is a Nucleotide Excision Repair (NER) gene.

24. The method of claim 16, wherein the at least one gene associated with DNA repair is a homologous recombination (HR) gene.

25. The method of claim 16, wherein the at least one gene associated with DNA repair comprises BRCA1, BRCA2, ATM, ATR, ERCC2, ERCC3, ERCC4, ERCC5, ERCC6, FANCD2, RAD51, or PALB2.

26. The method of claim 16, wherein the at least one biomarker comprises expression of RPA1, FANCE, FANCL, ERCC8/CSA, BRIP1, FANCF, MRE11A, BLM, ERCC3/XPB, FANCM, FANCB, FANCE, CHEK1, FANCI, ATM, ERCC4/XPG, ERCC6/CSB, CHEK2, ERCC2/XPD, OR ERCC3 genes.

27. The method of claim 15, wherein the at least one biomarker comprises expression of RPA1, FANCE, FANCL, BRIP1, FANCF, MRE11A, BLM, CHEK1, CHEK2, or ATM.

28. The method of claim 15, wherein the at least one biomarker comprises expression of PTGRI OR SDC4.

29. A method for treating a cancer in a subject in need thereof, comprising administering to the subject an effective amount of an anti-cancer agent, wherein the subject has a reduced expression level of a biomarker, a reduced expression of at least one gene associated with DNA repair, a reduced transcription level of at least one thereof, or any combination thereof, wherein the anti-cancer agent comprises illudin or an illudin analog.

30. The method of claim 29, wherein the reduced expression or transcription level is compared to a standard or control sample.

31. The method of claim 29, wherein the anti-cancer agent comprises illudin or an illudin analog.

32. The method of claim 29, wherein the cancer comprises a solid tumor.

33. The method of claim 32, wherein the solid tumor is a tumor of the breast, central nervous system, colon, skin, lung, ovary, prostate, or kidney.

34. The method of claim 29, wherein the cancer is a breast cancer, a central nervous system cancer, a colon cancer, a melanoma, a lung cancer, an ovarian cancer, a prostate cancer, or a renal cancer.

35. The method of claim 29, wherein the at least one gene associated with DNA repair is a DNA Damage Repair gene (DDRG).

36. The method of claim 29, wherein the at least one gene associated with DNA repair is a Nucleotide Excision Repair (NER) gene.

37. The method of claim 29, wherein the at least one gene associated with DNA repair is a homologous recombination (HR) gene.

38. The method of claim 29, wherein the at least one gene associated with DNA repair comprises BRCA1, BRCA2, ATM, ATR, ERCC2, ERCC3, ERCC4, ERCC5, ERCC6, FANCD2, RAD51, or PALB2.

39. The method of claim 29, wherein the at least one biomarker comprises expression of RPA1, FANCE, FANCL, ERCC8/CSA, BRIP1, FANCF, MRE11A, BLM, ERCC3/XPB, FANCM, FANCB, FANCE, CHEK1, FANCI, ATM, ERCC4/XPG, ERCC6/CSB, CHEK2, ERCC2/XPD, OR ERCC3 genes.

40. The method of claim 29, wherein the at least one biomarker comprises expression of RPA1, FANCE, FANCL, BRIP1, FANCF, MRE11A, BLM, CHEK1, CHEK2, or ATM.

41. The method of claim 29, wherein the at least one biomarker comprises expression of PTGRI OR SDC4.

42. The method of claim 29, wherein the transcription level is determined by measuring an amount of mRNA transcribed from the at least one gene associated with DNA repair.

43. A kit, for use in determining sensitivity of a specimen to an anti-cancer agent according to the method of any one of claims 1-14, wherein the kit comprises one or more reagents, standards, and instructions for use thereof, wherein the standards comprise one or more biomarkers or expression or transcription products, providing a threshold level, or a target level for screening sensitivity of the specimen to the anti-cancer agent, wherein the anti-cancer agent comprises illudin or an illudin analog.
